(19) United States
(12) Patent Application Publication
(10) Pub. No.: US 2009/0074829 A1
(43) Pub. Date: Mar. 19, 2009

(54) Novel Heparin Alternative Material and Method for Producing the Same

(76) Inventors: Masao Tanihara, Nara (JP); Kayo Hosoya, Hyogo (JP); Takeshi Takaha, Hyogo (JP); Junichi Takahara, Nara (JP); Michihiro Sunako, Osaka (JP)

Correspondence Address: WENDEROTH, LIND & PONACK, L.L.P., 2033 K STREET N. W., SUITE 800, WASHINGTON, DC, 20006-1021, US

(21) Appl. No.: 12/224,950

(22) PCT Filed: Mar. 13, 2007
(86) PCT No.: PCT/JP2007/054935

§ 371 Date: Sep. 10, 2008

(30) Foreign Application Priority Data
Mar. 14, 2006 (JP) 2006-069479

(51) Int. Cl. A61K 9/00; C08B 37/00; A61K 38/16; A61P 7/02

(52) U.S. Cl. 424/422; 536/123.12; 424/400; 514/12

(57) ABSTRACT

The object is to provide a novel heparin alternative material, which is excellent in safety and in vivo degradability. Disclosed are: a novel heparin alternative material, which comprises an enzymatically synthesized α-1,4-glucan derivative and has functions substituting those of heparin, such as an anticoagulation activity and functions of a material for storage or sustained release of a heparin-binding growth factor; a method for production of the substitute material; and a preparation or article for medical applications or a cosmetic produced using the heparin alternative material.

### TECHNICAL FIELD

The present invention relates to a novel heparin alternative material having functionalities such as anticoagulant function, storage function of heparin binding growth factor and controlled-release function. The present invention also relates to a method for producing the heparin alternative material, and a medical product, a medical material and a cosmetic using the heparin alternative material.

### BACKGROUND ART

In recent years, an artificial device and artificial apparatus such as extracorporeal circulation system or artificial kidney have been widely used. An artificial material which is included in the artificial device or artificial apparatus should be treated for preventing blood clotting in the artificial material's surface which is in contact with blood. Thus, demand of an anticoagulant agent has increased, and various anticoagulant agents are used. Heparin is well-known as an anticoagulant agent. Heparin has widely used as anticoagulant agent for medical apparatus containing intravital material or artificial material over long periods.

It is widely known that heparin also has stabilization function of heparin binding growth factor, and modulatory functions such as storage, controlled-release and assembly of heparin binding growth factor, in addition to the anticoagulant function. Typical heparin binding growth factor includes basic fibroblast growth factor (bFGF), hepatocellular growth factor (HGF) and bone morphogenetic protein (BMP) and so on. Heparin binding growth factor has intensive promotive effect of cell proliferation and cell differentiation against various cells, and is expected to be useful for wound treatment, broken born treatment, and regeneration and repair of blood vessel, nerves or liver. Non-patent literature 1 discloses a matrix having angiogenesis function and controlled-release function of basic fibroblast growth factor (bFGF), which is obtained by immersing heparin-covalently-bonded-alginate gel into the solution of basic fibroblast growth factor (bFGF).

Heparin is a medical material having important functionalities, however, has some drawbacks as follows.

(1) Heparin is sulfated mucopolysaccharide of animal origin, and is obtained by extraction and purification of intestine or lung of a mammal such as bovine, pig and lamp. Thus, it has possibilities of contamination of virus or prion. Furthermore, use of heparin derived from bovine's internal organ has been banned ever since prevalence of bovine spongiform encephalopathy (BSE). Safety of heparin derived from bovine is not ensured.

(2) Heparin promotes activity of antithrombin III which has anticoagulant activity in several-hundred-fold in the blood, and deactivates thrombin instantaneously. On the other hand, heparin is not degraded in the blood. Thus, heparin has a risk of decreasing blood clotting of its original function caused by heparin's anticoagulant function stability in the blood.

Methods for solving the two problems of heparin has been reported. One of the methods is to use a low-molecular-weight heparin. The low-molecular-weight heparin has weak binding activities for thrombin and may have a smaller risk of excessive bleeding. However, the low-molecular-weight heparin is also hardly degraded in the blood, and has a drawback of heparin's anticoagulant function stability in the blood. Furthermore, the method does not resolve the problem of the risk of contamination of virus or prion based on animal's internal organ.

Development for alternative polymer materials having heparin's function has been studied. For example, patent literature 1 discloses sulfated gellan, patent literature 2 discloses sulfated cellulose, and patent literature 3 discloses sulfonated fibroin or sericin. The alternative polymer materials in patent literatures 1 to 3 can resolve the problem of the risk of contamination of virus or prion based on animal's internal organ. However, the alternative polymer materials may be hardly degraded in the blood, and has a drawback of heparin's anticoagulant function stability in the blood. A problem of the alternative polymer materials' anticoagulant function stability in the blood and decreasing blood clotting of blood's original function is not resolved. Thus, development of heparin alternative material having high safety and excellent degradability in the blood has been desired, which can resolve the two drawbacks of heparin.

On the other hand, cosmetics containing cell growth factor such as epidermal growth factor (EGF) or fibroblast growth factor(FGF) to slow skin aging are commercially available. However, such cell growth factor has lower stability. Keeping cell-proliferative activity in cosmetics is difficult because cosmetics contain many components and tend to be stored for quite a while.

Patent literature 1: Japanese patent Kokai application No. 2004-2355

Patent literature 2: Japanese patent Kohyo application No. 2001-500184

Patent literature 3: Japanese patent kokai application No. Hei 9 (1997)-227402

Non patent literature 1: J. Biomed. Mater. Res., 216-221 (2001)

### DISCLOSURE OF INVENTION

### Problems to be Resolved by the Invention

Heparin used as anticoagulant agent is produced by extraction and purification of mammal such as bovine, pig and lamp, and has a safety problem. Furthermore, heparin has a risk of excessive bleeding because heparin is hardly degraded within a living body. Thus, heparin alternative material having high safety and excellent degradability in the blood after using anticoagulant agent has been desired. The object of the present invention is to solve the problems of heparin used for medical material and to provide a novel heparin alternative material having high safety and excellent degradability within a living body, method for producing the heparin alternative material, medical product, medical material and cosmetics using the heparin alternative material.

### Means of Solving the Problems

α-1,4-glucan is a polysaccharide having excellent degradability within a living body because α-1,4-glucan is degraded rapidly by α-amylase which is present in the blood and tissue of human body. Furthermore, α-1,4-glucan has excellent safety compared with animal-derived natural extract because α-1,4-glucan can be prepared by enzyme reaction. The inventors of the present invention have rigorously researched for achieving the above object, and eventually found that use of enzymatically synthesized α-1,4-glucan can provide novel heparin alternative material having high safety and excellent degradability within a living body. The inventors have also found that α-1,4-glucan having sulfate group and/or carboxyl group has better heparin-like function. Based on this finding, the inventors completed the present invention.

The present invention provides a heparin alternative material which is an enzymatically synthesized α-1,4-glucan derivative, which accomplishes the above objection.

The enzymatically synthesized α-1,4-glucan derivative may preferably have sulfonic acid group or carboxyl group.

The enzymatically synthesized α-1,4-glucan derivative may preferably have sulfonic acid group and carboxyl group.

The present invention also provides a heparin alternative material having anticoagulant function.

The present invention also provides an anticoagulant agent containing the enzymatically synthesized α-1,4-glucan derivative.

The present invention also provides a skin preparation for external use or a cosmetic which contains the enzymatically synthesized α-1,4-glucan derivative.

The present invention also provides a medical apparatus of which a surface is covered with the enzymatically synthesized α-1,4-glucan derivative.

The medical apparatus may preferably be any one selected from the group consisting of a blood collecting syringe, an artificial organ, a gel, a bind, a film, a sponge, a nonwoven fabric, a gauze, a bypass, a membrane.

The present invention also provides a heparin alternative material which has controlled-release function of a heparin binding growth factor.

The present invention also provides a composition for controlled-release of heparin binding growth factor containing a heparin binding growth factor and the enzymatically synthesized α-1,4-glucan derivative.

The present invention also provides a molded article for controlled-release of heparin binding growth factor containing a heparin binding growth factor and the enzymatically synthesized α-1,4-glucan derivative.

The present invention also provides a gel for controlled-release of heparin binding growth factor containing a heparin binding growth factor and a chemically-crosslinked enzymatically synthesized α-1,4-glucan derivative.

The present invention also provides a process for preparing the enzymatically synthesized α-1,4-glucan derivative for a heparin alternative material. One embodiment of the process includes the following:

introducing step of carboxyl group in which an enzymatically synthesized α-1,4-glucan is reacted with a dicarboxylic acid to introduce carboxyl group into the enzymatically synthesized α-1,4-glucan.

The another embodiment of the process further includes:

introducing step of sulfonic acid group in which an enzymatically synthesized α-1,4-glucan derivative having carboxyl group prepared in the introducing step of carboxyl group is reacted with a compound having amino group and sulfonic acid group to introduce sulfonic acid group into all or a part of the carboxyl group.

The present invention also provides an enzymatically synthesized α-1,4-glucan derivative obtainable from the process.

### EFFECT OF THE INVENTION

α-1,4-glucan has excellent safety compared with animal-derived natural extract because α-1,4-glucan can be prepared by enzyme reaction from plants. In addition, α-1,4-glucan is a polysaccharide having excellent degradability within a living body because α-1,4-glucan is degraded rapidly by α-amylase which is present in the blood and tissue of human body. The present invention provides novel heparin alternative material having excellent safety and excellent degradability within a living body by using enzymatically synthesized α-1,4-glucan, for example, obtained by sulfonation of α-1,4-glucan.

### BRIEF DESCRIPTION OF DRAWINGS

FIG. 1 is a graphic chart showing time course of degradation rate of fluorescent substrate, which indicates that the enzymatically synthesized α-1,4-glucan derivative having sulfonic acid group in the present invention has anticoagulant function.

FIG. 2 is a graphic chart showing degradation rate of fluorescent substrate in the 8th hour after start of test for blood clotting of the enzymatically synthesized α-1,4-glucan derivative having sulfonic acid group and/or carboxyl group.

### DETAILED DESCRIPTION OF THE PREFERRED EMBODIMENTS

### Explanation of Terms

When the “α-1,4-glucan” is used in the present specification, it means saccharide having D-glucose as a compositional unit, which has at least 2 or more of saccharide units connected with only α-1,4-glucoside linkage. The α-1,4-glucan is a linear chain molecule and is also called as linear chain glucan.

Further, the term “a polydispersity Mw/Mn” is a ratio of number average molecular weight Mn to weight average molecular weight Mw (that is, Mw÷Mn). A polymeric compound is not single in molecular weight irrespective of natural or non-natural origin except for a specific case such as protein and has breadth to a certain degree. Consequently, the polydispersity Mw/Mn is usually used in the art of polymer chemistry in order to show the polydispersity of the molecular weight of a polymeric compound. The polydispersity is an index of the breadth of molecular weight distribution of the polymeric compound. In case of a polymeric compound in which molecular weight is perfectly single, Mw/Mn is 1. And Mw/Mn is a value larger than 1 in accordance with the broadening of molecular weight distribution. The term “molecular weight” in the present specification indicates a weight average molecular weight (Mw) unless otherwise notified.

Enzymatically Synthesized α-1,4-glucan

The enzymatically synthesized α-1,4-glucan used in the present invention can be prepared by a known method in the art. The “enzymatically synthesized α-1,4-glucan” used in the present invention means α-1,4-glucan obtained by polymerizing saccharide by an enzyme reaction. The enzymatically synthesized α-1,4-glucan can be prepared by a known method in the art.

The example of the enzyme-synthesizing method includes a method using glucan phosphorylase (α-glucan phosphorylase, EC 2.4.1.1; usually called as phosphorylase). Phosphorylase is an enzyme catalyzing phosphorolysis reaction. One example of the enzyme-synthesizing method using phosphorylase is a method (hereinafter, called as a GP method) by which the glcosyl group of glucose-1-phosphate (hereinafter, called as G-1-P) being a substrate is transferred to, for example, maltoheptaose that is used as a primer. The GP method costs high in the industrial production of α-1,4-glucan because the G-1-P of a raw material is expensive, but has remarkable advantage that 100% linear chain α-1,4-glucan is obtained by successively polymerizing a saccharide unit with only α-1,4-glucoside. The GP method is well known in the art.

Another example of the enzyme-synthesizing method using phosphorylase is a method (hereinafter, called as the SP-GP method) by which α-1,4-glucan is enzymatically synthesized by using sucrose as a substrate and using, for example, maltooligosaccharide as a primer and by simultaneously acting this with sucrose phosphorylase EC 2.4.1.7 and glucan phosphorylase in the presence of inorganic phosphoric acid. The SP-GP method can produce 100% linear chain α-1,4-glucan with desired molecular weight in the same manner as the GP method and additionally, has advantage that production cost can be more lowered by using inexpensive sucrose as a raw material. The SP-GP method is well known in the art. The efficient production method of the SP-GP method is described, for example, in the pamphlet of International Patent Publication WO 02/097107. The enzymatically synthesized α-1,4-glucan used in the present invention may be produced in accordance with the method described in the pamphlet.

Further, the “primer” means a substance which functions as a starting material of glucan synthesis. Oligosaccharide can be used as the primer. It is preferable as the primer that maltooligosaccharide, for example, maltotriose, maltotetraose, maltopentaose, maltohexaose or amylose (e.g., α-1,4-glucan) may be used. As the primer, a single compound may be used or a mixture of 2 or more of compounds may be used. The average molecular weight of α-1,4-glucan obtained can be controlled by changing a ratio of the amount of primer and substrate. For example, α-1,4-glucan with a lower molecular weight can be obtained by increasing the amount of the primer. The α-1,4-glucan with a different average molecular weight can be thus easily prepared by changing the amount of the primer used.

On the other hand, although the AMSU method is also the synthesis method of α-1,4-glucan using enzyme, a polymerization degree of α-1,4-glucan obtained is extremely low (less than about 9 kDa) and the method is not suitable for the production of enzymatically synthesized α-1,4-glucan.

The enzymatically synthesized α-1,4-glucan obtained by the above-mentioned GP method and/or SP-GP method has the following characteristics:

(1) The molecular weight distribution is narrow (Mw/Mn is 1.1 or less). Accordingly, the control of physical properties is easy and α-1,4-glucan with stable performance can be obtained.
(2) The α-1,4-glucan having a desired molecular weight can be obtained by suitably controlling production conditions. Thereby, α-1,4-glucan having a molecular weight in accordance with the necessary physiological activity can be prepared.
(3) It is perfectly a linear chain and does not contain slight branched structure present in natural amylase fractionated from natural starch. That is, all unit is connected with an α-1,4-linkage and does not contain an α-1,6-linkage at all. Thereby, influence to physiological activity based on the branched structure portion can be excluded.
(4) Since it does not contain branched structure, there is no reaction disturbance by steric hindrance, and functional groups such as a sulfonic acid group and/or a carboxyl group can be introduced under more moderate conditions.
(5) Since it is constituted only by a glucose in the same manner as natural starch, there is no risk such as hazardousness originated in animal raw material. The α-1,4-glucan, all of its degraded intermediate and final degraded products have no hazardousness for a living body.

The molecular weight distribution of the enzymatically synthesized α-1,4-glucan used in the present invention may more preferably be 1.25 or less. α-1,4-glucan has various physical properties such as solubility relying on the molecular weight. Consequently, the reason is that physical properties such as solubility can be controlled well by using the enzymatically synthesized α-1,4-glucan having a narrow molecular weight distribution.

Further, the number average molecular weight of the enzymatically synthesized α-1,4-glucan used in the present invention may more preferably be 3 kDa to 2000 kDa. When the number average molecular weight is within the above-mentioned range, the solubility of α-1,4-glucan to a reaction solvent is enhanced at the time of introduction of a carboxyl group and a sulfonic acid group and viscosity is in an appropriate range; therefore, there is advantage that reaction efficiency is enhanced.

Further, as the preparation method of α-1,4-glucan other than the enzyme-synthesizing method, a method by cutoff from starch is mentioned. However, the molecular weight distribution of α-1,4-glucan fractionated from starch is broad and it contains branched structure. For example, the molecular weight distribution (Mw/Mn) of amylose contained in natural starch is as broad as 1.3 or more. Consequently, there is a problem that the physiological activity of α-1,4-glucan derivative obtained is indeterminate. The α-1,4-glucan obtained by such cutoff contains further branched structure. The branched structure becomes steric hindrance when a sulfonic acid group and/or a carboxyl group is introduced, and there is a problem that it intervenes the introduction of these groups.

Enzymatically Synthesized α-1,4-glucan Derivative

The “enzymatically synthesized α-1,4-glucan derivative” in the present specification means a compound obtained by introducing functional group in the enzymatically synthesized α-1,4-glucan. Examples of the functional group capable of being introduced in the α-1,4-glucan include carboxyl group, sulfonic acid group, and the like. Further, with respect to the description of a carboxyl group and a sulfonic acid group that the α-1,4-glucan derivative in the present specification has, a salt thereof (a salt of carboxylic acid, a salt of sulfonic acid and the like) are also included.

In the present invention, the enzymatically synthesized α-1,4-glucan derivative may preferably have at least one of the carboxyl group and sulfonic acid group. This is because the enzymatically synthesized α-1,4-glucan derivative having these groups has superior anticoagulant activity. The enzymatically synthesized α-1,4-glucan derivative having both of the carboxyl group and sulfonic acid group has more preferably superior anticoagulant activity and may preferably be used.

The substitution degree of the functional groups such as the carboxyl group and sulfonic acid group may more preferably be 0.5 to 2.8 in the enzymatically synthesized α-1,4-glucan derivative in the present invention. The “substitution degree” in the present specification represents an average substituted hydroxyl group number per an anhydrous glucose residual group in the α-1,4-glucan derivative. The hydroxyl groups of anhydrous glucose residual group are 3, and when all of the three groups are substituted by chemical modification, the substitution degree is 3, and when 2 hydroxyl groups are substituted in average, the substitution degree is 2. The substitution degree is mere an average value and its intermediate value may be assumed.

As one example of a method for preparing the enzymatically synthesized α-1,4-glucan derivative in the present invention, there is mentioned a method including a carboxyl group-introducing step in which a carboxyl group is introduced in the enzymatically synthesized α-1,4-glucan derivative by reacting the enzymatically synthesized α-1,4-glucan derivative with dicarboxylic acid. The carboxyl group can be more conveniently introduced in the enzymatically synthesized α-1,4-glucan derivative.

Examples of the dicarboxylic acid that can be used in the reaction include succinic acid, maleic acid, phthalic acid, oxalic acid, adipic acid, pimelic acid, suberic acid, azelaic acid, sebasic acid, undecane diacid, dodecanoic diacid, tridecanoic diacid, tetradecanoic diacid, pentadecanoic diacid, octadecanoic diacid, nonadecanoic diacid, eicosanoic diacid, and acid anhydrides such as, for example, succinic anhydride, maleic anhydride, phthalic anhydride, octenylsuccinic anhydride, dodecenyl succinic anhydride, hexadecenyl succinic anhydride and octadecenyl succinic anhydride. In the method, succinic anhydride, maleic anhydride, phthalic anhydride, octenylsuccinic anhydride and the like may more preferable be used as the dicarboxylic acid. The enzymatically synthesized α-1,4-glucan derivative having a carboxyl group superior in safety can be prepared under milder condition by using these dicarboxylic acids.

An amount of the dicarboxylic acid used in the carboxyl group-introducing step can be changed to various amounts in accordance with the substitution degree of the carboxyl group introduced in the enzymatically synthesized α-1,4-glucan. For example, when the α-1,4-glucan derivative with the dicarboxylic acid of 2 or more is prepared for three hydroxyl groups contained in a monosaccharide unit composing α-1,4-glucan, 2 to 9 mol of the dicarboxylic acid can be used for 1 mol of the monosaccharide unit. In the reaction of α-1,4-glucan derivative with the anhydride of the dicarboxylic acid, basic reagents such as diisopropylethylamine (DIPEA), triethylamine, pyridine and dimethylaminopyridine can be used for enhancing reactivity. Further, in the reaction of α-1,4-glucan with the dicarboxylic acid other than anhydride, a condensing agent, for example, carbodiimide condensing agents such as 1-ethyl-3-(3-dimethylaminopropyl)-carbodiimide hydrochloride (EDC.HCl), dicyclohexylcarbodimide (DCC) and diisopropylcarbodiimide (DIC); triazine condensing agents such as 4-(4,6-dimethoxy-1,3,5-triazin-2-yl)-4-methylmorpholine hydrochloride; and aluminum condensing agents, phosphonium condensing agents, dihydroquinone condensing agents and the like, can be used. In these reactions, dehydration condensing agents such as 1-hydroxybenzotriazole (HOBt), 1-hydroxy-7-azabenzotriazole (HOAt) and N-hydrosuccinimide (HOSu) may be used.

Specific examples of the solvent used in the carboxyl group-introducing step include ketone solvents such as methyl ethyl ketone, acetone, methyl isobutyl ketone and cyclohexanone; ether solvents such as diethyl ether, isopropyl ether, tetrahydrofuran, dioxane, ethylene glycol dimethyl ether, ethylene glycol diethyl ether, diethylene glycol dimethyl ether, diethylene glycol diethyl ether, propylene glycol monomethyl ether, anisole and phenetol; ester solvents such as ethyl acetate, butyl acetate, isopropyl acetate and ethylene glycol diacetate; and amide solvents such as dimethylformamide, diethylformamide, dimethylsulfoxide and N-methylpyrrolidone, etc. These solvents may be used alone or may be used in mixture.

The enzymatically synthesized α-1,4-glucan derivative having carboxyl groups is obtained by the carboxyl group-introducing step. The enzymatically synthesized α-1,4-glucan derivative superior in uniformity and safety and having carboxyl groups can be prepared under milder condition using the enzymatically synthesized α-1,4-glucan derivative by the above-mentioned carboxyl group-introducing step using the enzymatically synthesized α-1,4-glucan. Further, other functional group can be easily introduced by using the enzymatically synthesized α-1,4-glucan derivative having carboxyl groups for the sulfonic acid group-introducing step described below, and the like.

As another example of the method preparing the enzymatically synthesized α-1,4-glucan derivative, there is mentioned a method including a sulfonic acid group-introducing step in which the enzymatically synthesized α-1,4-glucan derivative having carboxyl groups obtained by the above-mentioned carboxyl group-introducing step is reacted with a compound containing an amino group and a sulfonic acid group and the sulfonic acid group is introduced in all or part of the carboxyl group.

Examples of the compound containing an amino group and a sulfonic acid group include aminomethanesulfonic acid, 2-aminoethanesulfonic acid, 3-aminopropanesulfonic acid, 4-amino-3-hydroxy-1-naphthalenesulfonic acid, 1-amino-8-naphthol-2,4-disulfonic acid, 2-aminobenzenesulfonic acid, 3-aminobenzenesulfonic acid, 4-aminobenzenesulfonic acid, and the like. As the compound containing amino group and sulfonic acid group used in the method, aminomethanesulfonic acid and 2-amnioethanesulfonic acid may more preferably be used. Further, the solvent used in the above-mentioned carboxyl group-introducing step can be similarly used in the sulfonic acid group-introducing step.

Further, in the method of the present invention, a compound other than the compound containing amino group and sulfonic acid group can be used by a similar method as the sulfonic acid group-introducing step. For example, the enzymatically synthesized α-1,4-glucan derivative having a nitro group can be easily obtained by reacting it by a similar method as the sulfonic acid group-introducing step, using a compound containing amino group and nitro group such as nitroethaneamine. Similarly, the introduction of a thiol group by a thiol group-introducing step such as cysteamine and the introduction of a phosphoric acid group by a phosphoric acid group-introducing step such as aminoethanephosphonic acid can be carried out. Further, a crosslinking structure can be formed by reacting it by a similar method as the sulfonic acid-introducing step, using a diamine compound.

In the carboxyl group-introducing step, carboxyl group is coupled with amino group by condensation, by reacting carboxyl groups that the enzymatically synthesized α-1,4-glucan derivative has, with the compound containing amino group and sulfonic acid group, and thereby, the sulfonic acid group is introduced. In the reaction, there can be used a condensing agent, for example, carbodiimide condensing agents such as 1-ethyl-3-(3-dimethylaminolpropyl)-carbodiimide hydrochloride (EDC.HCl), dicyclohexylcarbodimide (DCC) and diisopropylcarbodiimide (DIC); triazine condensing agents such as 4-(4,6-dimethoxy-1,3,5-triazin-2-yl)-4-methylmorpholine hydrochloride; further; and aluminum condensing agents, phosphonium condensing agents, dihydroquinone condensing agents and the like. Further, as a condensing additive, 1-hydroxybenzotriazole (HOBt), 1-hydroxy-7-azabenzotriazole (HOAt), N-hydrosuccinimide (HOSu) and the like can be used.

An amount of the sulfonic acid group to be introduced in the sulfonic acid group-introducing step can be easily changed by changing the amount of the compound containing amino group and sulfonic acid group to be used. For example, sulfonic acid group can be introduced into all of carboxyl groups that the enzymatically synthesized α-1,4-glucan derivative has by using the amount of 1 mol equivalent to or more of the compound containing an amino group and a sulfonic acid group based on 1 mol of the carboxyl group that the enzymatically synthesized α-1,4-glucan derivative has. Further, for example, the enzymatically synthesized α-1,4-glucan derivative having both of carboxyl group and sulfonic acid group can be easily prepared by introducing the amount of less than 1 mol equivalent of the compound containing amino group and sulfonic acid group based on 1 mol of carboxyl group that the enzymatically synthesized α-1,4-glucan derivative has. Further, the introduction ratio of two substituents can be also changed. Further, it has been found from the experiment by the present inventors that the enzymatically synthesized α-1,4-glucan derivative having both of a carboxyl group and a sulfonic acid group has very superior heparin alternative function; therefore, it is a more preferable derivative. Further, since it has carboxyl group, the introduction of other functional groups and crosslinking reaction are easy and physiological activity and physical properties can be changed.

Further, as the method of producing the α-1,4-glucan derivative having sulfonic acid group, for example, there is mentioned a method of reacting ethyleneimine with the hydroxyl group of α-1,4-glucan to prepare amino ethyl-etherified glucan and then, reacting a sulfonic acidifying reagent such as chlorosulfonic acid or anhydrous sulfonic acid to introduce sulfonic acid group, other than the above-mentioned production method. The α-1,4-glucan derivative in the present invention can be also prepared using such a production method. However, such method is complicated in steps and further, and the easy adjustment of the substitution degree of the sulfonic acid group or carboxyl group that the α-1,4-glucan derivative has may be difficult.

By the way, as a method of introducing sulfonic acid group into the hydroxyl group of saccharide chain, there is generally mentioned a method of substituting a hydroxyl group with a sulfonic acid group, using sulfonating agents such as anhydrous sulfuric acid, sulfuric acid and chlorosulfonic acid. However, the reaction is a method of using very reactive sulfonating agents such as sulfuric acid or chlorosulfonic acid. These sulfonating agents not only may substitute the hydroxyl group of saccharide chain with a sulfuric acid group, but also may generate side reaction in accordance with the scission of the glycoside linkage of saccharide chain and the destruction of saccharide structure. By contrast, the method of the present invention is a very superior method by which more functional groups such as carboxyl group and/or sulforic acid group can be introduced under very mild condition. The method of the present invention can easily introduce a functional group under very mild condition without accompanying the scission of saccharide chain and the destruction of saccharide structure.

The enzymatically synthesized α-1,4-glucan derivative in the present invention has also further the advantages described below in addition to the above-mentioned advantages of the enzymatically synthesized α-1,4-glucan.

(1) Since the enzymatically synthesized α-1,4-glucan used for the preparation of the enzymatically synthesized α-1,4-glucan derivative does not contain branched structure, it has not steric hindrance. Consequently, it can prepare the enzymatically synthesized α-1,4-glucan derivative having much more carboxyl groups and/or sulfonic acid groups.
(2) The physiological activity and degradation time of the enzymatically synthesized α-1,4-glucan derivative can be controlled by adjusting a kind and an amount of functional groups introduced in α-1,4-glucan.

Anticoagulant Activity

The enzymatically synthesized α-1,4-glucan derivative obtained by the above-description is used as a heparin alternative material. One of the heparin alternative functions that the enzymatically synthesized α-1,4-glucan derivative in the present invention has is anticoagulant activity. The enzymatically synthesized α-1,4-glucan derivative obtained by the present invention has anticoagulant activity and can be used as an anticoagulant drug formulation. Further, a medical apparatus can have anticoagulant activity by coating the enzymatically synthesized α-1,4-glucan derivative of the present invention on the medical apparatus. Examples of the medical apparatus include a blood collecting syringe, an artificial internal organ, a gel, a bind, a film, a sponge, a non-woven fabric, a gauze, a bypath, a membrane, and the like.

As the method of coating the enzymatically synthesized α-1,4-glucan derivative on a medical apparatus, for example, there are mentioned a method of binding the enzymatically synthesized α-1,4-glucan derivative in the present invention by covalent bond, electrostatic interaction, hydrogen bonding and the like, using a coating composition that forms a living body adaptable polymers such as poly(2-methoxyethyl acrylate) (PMEA) and poly(2-hydroxyethyl methacrylate) (PHEMA), etc. The enzymatically synthesized α-1,4-glucan derivative in the present invention can be coated on the surface of the above-mentioned medical apparatus by the method.

Controlled-Release Function of Heparin Binding Growth Factor

Other function of heparin-like function which the enzymatically synthesized α-1,4-glucan derivative according to the present invention has is controlled-release function of heparin binding growth factor. The heparin alternative material containing the enzymatically synthesized α-1,4-glucan derivative and heparin binding growth factor has function of sustained-releasing of heparin binding growth factor. Introducing the heparin alternative material within a living body for sustained-releasing heparin binding growth factor can provide cell growth-promoting activity and/or differentiation-stimulated activity within a living body. An example of the heparin binding growth factor which can be contained in the heparin alternative material includes basic fibroblast growth factor (bFGF), hepatocellular growth factor (HGF) and bone morphogenetic protein (BMP).

Preparation of a Composition Containing the enzymatically synthesized α-1,4-glucan derivative and the heparin binding growth factor can provide a composition for controlled-release of heparin binding growth factor. Molding of the composition for controlled-release of heparin binding growth factor can provide a molded article for controlled-release of heparin binding growth factor. The molded article for controlled-release of heparin binding growth factor has heparin-like function. Introducing the molded article for controlled-release of heparin binding growth factor within a living body for extended-releasing heparin binding growth factor can provide cell growth-promoting activity and/or differentiation-stimulated activity within a living body.

The enzymatically synthesized α-1,4-glucan derivative contained in the heparin alternative material, the composition for controlled-release of heparin binding growth factor and the molded article for controlled-release of heparin binding growth factor according to the present invention may be chemically-crosslinked. Chemically cross-linking of the enzymatically synthesized α-1,4-glucan derivative provides three-dimension structure for the derivative, in which the derivative can have excellent controlled-release function. A method for chemically cross-linking of the enzymatically synthesized α-1,4-glucan derivative includes, for example, making a cross-linked structure using a cross-linking agent such as ethylenediamine 2N-hydroxysuccinimide salt (EDA-2HOSu), epichlorohydrin and glutaraldehyde. Usage example of the chemically-crosslinked enzymatically synthesized α-1,4-glucan derivative includes a gel for controlled-release of heparin binding growth factor containing the heparin binding growth factor and the chemically-crosslinked enzymatically synthesized α-1,4-glucan derivative.

The enzymatically synthesized α-1,4-glucan derivative according to the present invention may be contained in a skin preparation for external use or a cosmetic. The enzymatically synthesized α-1,4-glucan derivative according to the present invention has anti-inflammatory function, blood circulation promotion function, assist function of water retentivity of the skin, as well as heparin alternative function. Using the enzymatically synthesized α-1,4-glucan derivative according to the present invention can provide an skin preparation for external use or a cosmetic having anti-inflammatory function, blood circulation promotion function and assist function of water retentivity of the skin. Furthermore, preparing a skin preparation for external use or a cosmetic containing the enzymatically synthesized α-1,4-glucan derivative according to the present invention and the heparin binding growth factor can provide long-term retention of activity of the growth factor, which can provide effective action for the skin. A specific example of the cosmetic includes, for example, skin-care cosmetic and scalp cosmetic.

### EXAMPLE

### Production Example 1

### Synthesis of α-1,4-glucan Having Average Weight Molecular of 5 kDa

Four-liter of aqueous solution containing sucrose (3%), sucrose phosphorylase (1200 U/L), glucan phosphorylase (1200 U/L), inorganic phosphoric acid (15 mM), TETRUP H (manufactured by HAYASHIBARA SHOJI, INC.) (9000 μM) was subjected to an enzymatic reaction at 45° C. for 8 hours. After the reaction was completed, the reaction mixture was cooled to 10° C. for 14 hours to precipitate α-1,4-glucan. The resulted precipitate was dried by a hot air dryer to obtain α-1,4-glucan (about 50 g). Thus obtained α-1,4-glucan has a weight-average molecular weight of about 5 kDa and 1.05 of polydispersity Mw/Mn.

### Production Example 2

### Synthesis of α-1,4-glucan Having Average Weight Molecular of 30 kDa

Four-liter of aqueous solution containing sucrose (3%), sucrose phosphorylase (1200 U/L), glucan phosphorylase (1200 U/L), inorganic phosphoric acid (15 mM), TETRUP H (manufactured by HAYASHIBARA SHOJI, INC.) (1500 μM) was subjected to an enzymatic reaction at 45° C. for 8 hours. After the reaction was completed, the reaction mixture was cooled to 10° C. for 14 hours to precipitate α-1,4-glucan. The resulted precipitate was dried by a hot air dryer to obtain α-1,4-glucan (about 50 g). Thus obtained α-1,4-glucan has a weight-average molecular weight of about 30 kDa and 1.02 of polydispersity Mw/Mn.

### Production Example 3

### Synthesis of α-1,4-glucan Having Average Weight Molecular of 90 kDa

Four-liter of aqueous solution containing sucrose (3%), sucrose phosphorylase (1200 U/L), glucan phosphorylase (1200 U/L), inorganic phosphoric acid (15 mM), TETRUP H (manufactured by HAYASHIBARA SHOJI, INC.) (500 μM) was subjected to an enzymatic reaction at 45° C. for 8 hours. After the reaction was completed, the reaction mixture was cooled to 10° C. for 14 hours to precipitate α-1,4-glucan. The resulted precipitate was dried by a hot air dryer to obtain α-1,4-glucan (about 45 g). Thus obtained α-1,4-glucan has a weight-average molecular weight of about 90 kDa and 1.03 of polydispersity Mw/Mn.

### Production Example 4

### Synthesis of α-1,4-glucan Having Average Weight Molecular of 500 kDa

Four-liter of aqueous solution containing sucrose (6%), sucrose phosphorylase (1200 U/L), glucan phosphorylase (1200 U/L), inorganic phosphoric acid (30 mM), TETRUP H (manufactured by HAYASHIBARA SHOJI, INC.) (80 μM) was subjected to an enzymatic reaction at 45° C. for 8 hours. After the reaction was completed, ethanol was added to the reaction mixture for adjusting the ethanol content of 33% to precipitate α-1,4-glucan. The resulted precipitate was dried by a hot air dryer to obtain α-1,4-glucan (about 95 g). Thus obtained α-1,4-glucan has a weight-average molecular weight of about 500 kDa and 1.03 of polydispersity Mw/Mn.

### Production Example 5

### Synthesis of α-1,4-glucan Having Average Weight Molecular of 1000 kDa

Four-liter of aqueous solution containing sucrose (6%), sucrose phosphorylase (1200 U/L), glucan phosphorylase (1200 U/L), inorganic phosphoric acid (30 mM), TETRUP H (manufactured by HAYASHIBARA SHOJI, INC.) (18 μM) was subjected to an enzymatic reaction at 45° C. for 8 hours. After the reaction was completed, ethanol was added to the reaction mixture for adjusting the ethanol content of 33% to precipitate α-1,4-glucan. The resulted precipitate was dried by a hot air dryer to obtain α-1,4-glucan (about 90 g). Thus obtained α-1,4-glucan has a weight-average molecular weight of about 1000 kDa and 1.02 of polydispersity Mw/Mn.

### Example 1

### Preparation of α-1,4-glucan Derivative Having Carboxyl Group

The enzymatically synthesized α-1,4-glucan (2 g) having an average molecular weight of 5 kDa obtained by production example 1 was dissolved in 40 ml of dimethylsulfoxide (DMSO). 6.3 ml of N,N-diisopropylethylamine (DIPEA) and 3.6 g of succinic anhydride were added thereto and mixed at 20° C. for one hour. After the reaction was completed, the resultant was diluted using 160 ml of ultrapure water and was dialyzed. After dialysis for three days, the resultant was freeze-dried to obtain α-1,4-glucan derivative having carboxyl group. An infrared absorption spectrum of the resulting sample indicates new absorption peak in 1730-1735 cm⁻¹ originated in ester group. Thus, introduction of carboxyl group was confirmed by the spectrum.

### Example 2

### Preparation of α-1,4-glucan Derivative Having Carboxyl Group and Sulfonic Acid Group

α-1,4-glucan derivative (2 g) obtained by example 1 was dissolved in 138 ml of DMSO. 0.64 g of taurine (0.5 molar equivalent for 1 mole of carboxyl group in the resultant α-1,4-glucan derivative) was added and stirred. After dissolving, 0.59 g of N-hydroxysuccinimide (HOSu) (0.5 molar equivalent for 1 mole of carboxyl group in the resultant α-1,4-glucan derivative) and 0.89 ml of N,N-diisopropylethylamine (DIPEA) (0.5 molar equivalent for 1 mole of carboxyl group in the resultant α-1,4-glucan derivative) were added and stirred. 1.96 g of 1-ethyl-3-(3-dimethylaminopropyl)-carbodiimide hydrochloride(EDC.HCl) (1.0 molar equivalent for 1 mole of carboxyl group in the resultant α-1,4-glucan derivative) was added and stirred overnight at 20° C. After the reaction was completed, the resultant was diluted using 552 ml of ultrapure water and was dialyzed. After dialysis for three days, the resultant was freeze-dried to obtain α-1,4-glucan derivative having carboxyl group and sulfonic acid group. An infrared absorption spectrum of the resulting sample indicates absorption peak in 1730-1735 cm⁻¹ originated in ester group. Thus, presence of carboxyl group was confirmed by the spectrum.

Furthermore, the infrared absorption spectrum indicates new absorption peaks in 1640-1660 cm⁻¹ originated in amide I, in 1560-1565 cm⁻¹ originated in amide II, and in 1037-1041 cm⁻¹, 1174-1181 cm⁻¹ and 1211-1215 cm⁻¹ originated in sulfonic acid. Thus, introduction of sulfonic acid group was confirmed by the spectrum.

### Example 3

### Preparation of α-1,4-glucan Having Sulfonic Acid Group

Two gram of α-1,4-glucan derivative obtained by example 1 was dissolved in 138 ml of DMSO. 3.84 g of taurine (3.0 molar equivalent for 1 mole of carboxyl group in the resultant α-1,4-glucan derivative) was added and stirred. After dissolving, 3.54 g of N-hydroxysuccinimide (HOSu) (3.0 molar equivalent for 1 mole of carboxyl group in the resultant α-1,4-glucan derivative) and 5.36 ml of N,N-diisopropylethylamine (DIPEA) (3.0 molar equivalent for 1 mole of carboxyl group in the resultant α-1,4-glucan derivative) were added and stirred. 11.8 g of 1-ethyl-3-(3-dimethylaminopropyl)carbodiimide hydrochloride (EDC.HCl) (6.0 molar equivalent for 1 mole of carboxyl group in the resultant α-1,4-glucan derivative) was added and stirred overnight at 20° C. After the reaction was completed, the resultant was diluted using 552 ml of ultrapure water and was dialyzed. After dialysis for three days, the resultant was freeze-dried to obtain α-1,4-glucan derivative having sulfonic acid group. An infrared absorption spectrum of the resulting sample amide indicates new absorption peaks in 1640-1660 cm⁻¹ originated in amide 1, 1560-1565 cm⁻¹ originated in amide II, and 1037-1041 cm⁻¹, 1174-1181 cm⁻¹ and 1211-1215 cm⁻¹ originated in sulfonic acid. Thus, introduction of sulfonic acid group was confirmed by the spectrum.

### Example 4

### Preparation of α-1,4-glucan Derivative Having Sulfonic Acid Group

A similar procedure of example 1 was performed by using 2 g of enzymatically synthesized α-1,4-glucan having an average molecular weight of 30 kDa obtained by production example 2 instead of enzymatically synthesized α-1,4-glucan having an average molecular weight of 5 kDa to obtain α-1,4-glucan derivative having carboxyl group. Next, similar procedure of example 3 was performed by using the resultant α-1,4-glucan derivative having carboxyl group instead of α-1,4-glucan derivative obtained by example 1 to obtain α-1,4-glucan derivative having sulfonic acid group.

### Example 5

### Preparation of α-1,4-glucan Derivative Having Carboxyl Group

A similar procedure of example 1 was performed by using 2 g of enzymatically synthesized α-1,4-glucan having an average molecular weight of 90 kDa obtained by production example 3 instead of enzymatically synthesized α-1,4-glucan having an average molecular weight of 5 kDa to obtain α-1,4-glucan derivative having carboxyl group.

### Example 6

### Preparation of α-1,4-glucan Derivative Having Carboxyl Group and Sulfonic Acid Group

A similar procedure of example 2 was performed by using 2 g of α-1,4-glucan derivative having an average molecular weight of 90 kDa obtained by example 5 instead of enzymatically synthesized α-1,4-glucan obtained by example 1 to obtain α-1,4-glucan derivative having carboxyl group and sulfonic acid group.

### Example 7

### Preparation of α-1,4-glucan Derivative Having Sulfonic Acid Group

A similar procedure of example 3 was performed by using 2 g of α-1,4-glucan derivative having an average molecular weight of 90 kDa obtained by example 5 instead of α-1,4-glucan derivative obtained by example 1 to obtain α-1,4-glucan derivative having sulfonic acid group.

### Example 8

### Preparation of α-1,4-glucan Derivative Having Sulfonic Acid Group

A similar procedure of example 1 was performed by using 2 g of enzymatically synthesized α-1,4-glucan having an average molecular weight of 500 kDa obtained by production example 4 instead of enzymatically synthesized α-1,4-glucan having an average molecular weight of 5 kDa to obtain α-1,4-glucan derivative having carboxyl group.

Next, similar procedure of example 3 was performed by using the resultant α-1,4-glucan derivative having carboxyl group instead of α-1,4-glucan derivative obtained by example 1 to obtain α-1,4-glucan derivative having sulfonic acid group.

### Example 9

### Preparation of α-1,4-glucan Derivative Having Carboxyl Group

A similar procedure of example 1 was performed by using 2 g of enzymatically synthesized α-1,4-glucan having an average molecular weight of 1000 kDa obtained by production example 5 instead of enzymatically synthesized α-1,4-glucan having an average molecular weight of 5 kDa to obtain α-1,4-glucan derivative having carboxyl group.

### Example 10

### Preparation of α-1,4-glucan Derivative Having Carboxyl Group and Sulfonic Acid Group

A similar procedure of example 2 was performed by using 2 g of enzymatically synthesized α-1,4-glucan having an average molecular weight of 1000 kDa obtained by example 9 instead of enzymatically synthesized α-1,4-glucan obtained by example 1 to obtain α-1,4-glucan derivative having carboxyl group and sulfonic acid group.

### Example 11

### Preparation of α-1,4-glucan Derivative Having Sulfonic Acid Group

A similar procedure of example 3 was performed by using 2 g of enzymatically synthesized α-1,4-glucan having an average molecular weight of 1000 kDa obtained by example 9 instead of α-1,4-glucan derivative obtained by example 1 to obtain α-1,4-glucan derivative having sulfonic acid group.

The following Tables 1 to 4 indicates degree of substitution of carboxyl group and/or sulfonic acid group in the α-1,4-glucan derivatives according to the present invention. These degrees of substitution of α-1,4-glucan derivative were measured using nuclear magnetic resonance (NMR). Deuterium-substituted each samples was dissolved in DMSO-d6, and ¹H-NMR of each sample was observed by NMR (ECP600, manufactured by JEOL Ltd.). Degree of substitution was calculated by area ratio of α-1,4-glucan-origin proton, carboxyl group-origin proton, and sulfonic acid group-origin proton in the observed spectrum. The degrees of substitution of the α-1,4-glucan derivatives are shown in the following Tables.

TABLE 1

| | Exam- | Exam- | Exam- | Exam- | Exam- |
|---|---|---|---|---|---|
| | ple 1 | ple 2 | ple 3 | ple 4 | ple 5 |
| Average | 5 | 5 | 5 | 30 | 90 |
| molecular | | | | | |
| weight (kDa) | | | | | |
| polydispersity | 1.05 | 1.05 | 1.05 | 1.02 | 1.03 |
| Mw/Mn | | | | | |
| Degree of | 2.3 | 1.6 | 0 | * | 1.3 |
| substitution | | | | | |
| of carboxyl | | | | | |
| group | | | | | |
| Degree of | — | 0.7 | 2.3 | * | — |
| substitution | | | | | |
| of sulfonic | | | | | |
| acid group | | | | | |

TABLE 2

| | Exam- | Exam- | Exam- | Exam- | Exam- |
|---|---|---|---|---|---|
| | ple 6 | ple 7 | ple 8 | ple 9 | ple 10 |
| Average | 90 | 90 | 500 | 1000 | 1000 |
| molecular | | | | | |
| weight (kDa) | | | | | |
| polydispersity | 1.03 | 1.03 | 1.03 | 1.02 | 1.02 |
| Mw/Mn | | | | | |
| Degree of | 0.9 | 0.0 | * | 1.3 | 1.0 |
| substitution | | | | | |
| of carboxyl | | | | | |
| group | | | | | |
| Degree of | 0.4 | 1.3 | * | — | 0.3 |
| substitution | | | | | |
| of sulfonic | | | | | |
| acid group | | | | | |

TABLE 3

| | example 11 |
|---|---|
| Average molecular weight (kDa) | 1000 |
| polydispersity Mw/Mn | 1.02 |
| Degree of substitution of carboxyl group | 0 |
| Degree of substitution of sulfonic acid group | 1.3 |

* not determined

### Example 12

### Test and Result of Anticoagulant Activity of α-1,4-glucan Derivative Having Sulfonic Acid Group

Each of α-1,4-glucan derivative (average molecular weight: 30 kDa) having sulfonic acid group obtained by example 4 (0.01 g) and α-1,4-glucan derivative (average molecular weight: 500 kDa) having sulfonic acid group obtained by example 8 (0.01 g) was dissolved in 1 ml of physiological saline solution. On the other hand, blood plasma (dryhemato blood-clotting control blood plasma level 1, manufactured by Wako Pure Chemical Industries, Ltd.) was dissolved in 0.5 ml of ultrapure water to obtain a blood plasma solution. 10 μl of Boc-VPR-MCA (10 mM solution in DMSO) was diluted by 90 μl of tris buffer to obtain a 10-fold diluted Boc-VPR-MCA solution. 5 ml of AMC (10 mM solution in DMSO) was diluted with 45 μl of tris buffer to obtain a tris buffer solution. 0.1 g of calcium chloride was dissolved in 10 ml of ultrapure water to obtain a solution containing 1% by weight of calcium chloride.

Thus obtained solutions were mixed in the following ratio.

| Sample solution | | |
|---|---|---|
| Tris buffer solution | 300 | μl |
| Solution of α-1,4-glucan | 100 | μl |
| derivative having sulfonic acid | | |
| group | | |
| Blood plasma solution | 50 | μl |
| 10-fold diluted Boc-VPR-MCA | 10 | μl |
| solution | | |
| Solution containing 1% by | 50 | μl |
| weight of calcium chloride | | |

| Heparin solution | | |
|---|---|---|
| Tris buffer solution | 300 | μl |
| Heparin solution or low- | 100 | μl |
| molecular-weight heparin | | |
| solution | | |
| Blood plasma solution | 50 | μl |
| 10-fold diluted Boc-VPR-MCA | 10 | μl |
| solution | | |
| Solution containing 1% by | 50 | μl |
| weight of calcium chloride | | |

*heparin: pig intestinal mucosal-origin heparin, manufactured by Sigma
*low-molecular-weight heparin: pig intestinal mucosal-origin heparin having molecular weight of about 6000 Da, manufactured by Sigma

| Control solution | |
|---|---|
| Tris buffer solution | 400 μl |
| Blood plasma solution | 50 μl |
| 10-fold diluted Boc-VPR-MCA | 10 μl |
| solution | |
| Solution containing 1% by | 50 μl |
| weight of calcium chloride | |

| Standard solution | |
|---|---|
| Tris buffer solution | 500 μl |
| 10-fold diluted AMC solution | 10 μl |

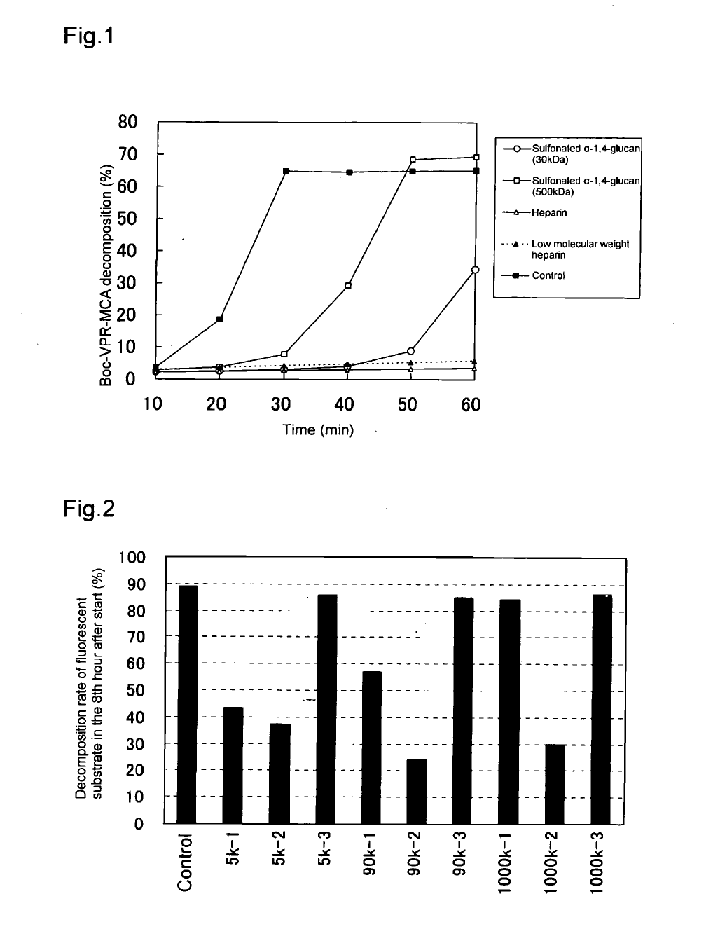

Each well of 96-well plate (manufactured by Nunc) was filled with 100 μl of the above solution, and fluorescence emission at 465 nm based on excitation light at 360 nm of aminomethyl coumarin (AMC) was observed by Plate leader manufactured by Tecan (SPECTRA Fluor Plus), at ten-minute interval for one hour (shown in FIG. 1). When thrombin is activated, Boc-VPR-MCA that is substrate of thrombin is hydrolyzed and AMC becomes free. Thrombin activity was determined by measuring fluorescence intensity of AMC. FIG. 1 clearly indicates that the α-1,4-glucan derivatives having sulfonic acid group according to the present invention inhibit thrombin activity. Inhibition of thrombin activity can prevent formation of fibrin clot based on fibrinogen in blood plasma. The result of this example confirmed that the α-1,4-glucan derivative having sulfonic acid group according to the present invention has anticoagulant function.

### Example 13

### Test and Result of Anticoagulant Activity of α-1,4-glucan Derivative According to the Present Invention

Each sample (1 mg) shown in Table 4 was dissolved in 1 ml of tris buffer. Blood plasma (dryhemato blood-clotting control blood plasma level 1, manufactured by Wako Pure Chemical Industries, Ltd.) was dissolved in 0.5 ml of ultrapure water and was diluted by adding 2 ml of tris buffer. A similar procedure of example 12 other than the above was performed for determining anticoagulant activity. FIG. 2 shows hydrolysis rate of Boc-VPR-MCA as a substrate in the 8th hour after start of test. Shown in FIG. 2, the result of this example confirmed that each of the α-1,4-glucan derivatives having both sulfonic acid group and carboxyl group, i.e., 5k-2, 90k-2 and 1000k-2, has lower degradation rate and better anticoagulant activity in various molecular weight of α-1,4-glucan.

TABLE 4

| | | Number | | |
|---|---|---|---|---|
| | | average | Degree of | Degree of |
| | | molecular | substitution | substitution |
| | Example | weight | of carboxyl | of sulfonic |
| Sample | No. | (kDa) | group | acid group |
| 5k-1 | 1 | 5 | 2.3 | 0.0 |
| 5k-2 | 2 | 5 | 1.6 | 0.7 |
| 5k-3 | 3 | 5 | 0.0 | 2.3 |
| 90k-1 | 5 | 90 | 1.3 | 0.0 |
| 90k-2 | 6 | 90 | 0.9 | 0.4 |
| 90k-3 | 7 | 90 | 0.0 | 1.3 |
| 1000k-1 | 9 | 1000 | 1.3 | 0.0 |
| 1000k-2 | 10 | 1000 | 1.0 | 0.3 |
| 1000k-3 | 11 | 1000 | 0.0 | 1.3 |

### Example 14

### Controlled-Release Substrate of Heparin Binding Growth Factor

### 14-1: Preparation of Ethylenediamine 2N-hydroxysuccinimide Salt (EDA.2HOSu)

N-hydroxysuccinimide (HOSu) (2.3 g) was dissolved in 150 ml of ethyl acetate. To the mixture, 0.6 g of ethylenediamine (EDA) dissolved in 10 ml of ethyl acetate was added dropwise at room temperature in stirring. The mixture was further stirred for one hour after the dropping. Precipitated crystal was recrystallized using hot methanol to obtain 2.0 g of ethylenediamine 2N-hydroxysuccinimide salt (EDA.2HOSu).

### 14-2: Preparation of Controlled-Release Substrate of Heparin Binding Growth Factor

Two gram of α-1,4-glucan derivative having carboxyl group obtained by example 9 was dissolved in 138 ml of DMSO. Then, 0.5 g of taurine was added thereto and mixed. After dissolving, 0.75 g of N-hydroxysuccinimide (HOSu) and 1.1 ml of N,N-diisopropylethylamine (DIPEA) were added thereto and stirred. 2.6 g of 1-ethyl-3-(3-dimethylaminopropyl) carbodiimide hydrochloride (EDC.HCl) was added and stirred overnight at 20° C. After the reaction was completed, it was diluted using 552 ml of ultrapure water and was dialyzed. After dialysis for three days, the resultant was freeze-dried. An infrared absorption spectrum of the resulting sample confirmed that sulfonic acid group is introduced to the α-1,4-glucan derivative. To the resulting enzymatically synthesized α-1,4-glucan having sulfonic acid group (1 g), 40 ml of ultrapure water was added and was dissolved.

Resultant ethylenediamine 2N-hydroxysuccinimide salt (EDA.2HOSu) (0.44 g) was dissolved. After dissolving, 3.2 g of 1-ethyl-3-(3-dimethylaminopropyl) carbodiimide hydrochloride (EDC.HCl) was added and stirred at room temperature. The mixture was poured into a teflon-coated stainless-steel tray (13 cm×17 cm) and left at rest at about 25° C. for 48 hours to obtain cross-linking material. The resultant was washed adequately by an aqueous solution containing 2.5 mM of calcium chloride and 143 mM of sodium chloride. After that, the resultant was washed several times by ultrapure water and was freeze-dried to obtain a xerogel-like controlled-release substrate.

### INDUSTRIAL APPLICABILITY

The novel heparin alternative material which is an enzymatically synthesized α-1,4-glucan derivative according to the present invention has functionalities of anticoagulant function, storage function of heparin binding growth factor and controlled-release function. The heparin alternative material can be used as a medical product, medical material or cosmetics.

1-15. (canceled)

16. A heparin alternative material of an enzymatically synthesized α-1,4-glucan derivative having sulfonic acid group and carboxyl group wherein,
the carboxyl group in the enzymatically synthesized α-1,4-glucan derivative is carboxyl group which is introduced by reacting an enzymatically synthesized α-1,4-glucan derivative with a dicarboxylic acid.

17. A heparin alternative material according to claim 16 wherein the dicarboxylic acid is a succinic acid.

18. A heparin alternative material according to claim 16 or 17 wherein the heparin alternative material has anticoagulant function.

19. An anticoagulant agent comprising the enzymatically synthesized α-1,4-glucan derivative of claim 16 or 17.

20. A skin preparation for external use or a cosmetic which comprises the enzymatically synthesized α-1,4-glucan derivative of claim 16 or 17.

21. A medical apparatus of which a surface is covered with the enzymatically synthesized α-1,4-glucan derivative of claim 16 or 17.

22. A medical apparatus according to claim 21 which is any one selected from the group consisting of a blood collecting syringe, an artificial organ, a gel, a bind, a film, a sponge, a nonwoven fabric, a gauze, a bypass, a membrane.

23. A heparin alternative material according to claim 16 or 17 which has controlled-release function of a heparin binding growth factor.

24. A composition for controlled-release of heparin binding growth factor comprising a heparin binding growth factor and the enzymatically synthesized α-1,4-glucan derivative of claim 16 or 17.

25. A molded article for controlled-release of heparin binding growth factor comprising a heparin binding growth factor and the enzymatically synthesized α-1,4-glucan derivative of claim 16 or 17.

26. A gel for controlled-release of heparin binding growth factor comprising a heparin binding growth factor and an enzymatically synthesized α-1,4-glucan derivative obtained by chemically-crosslinked of the enzymatically synthesized α-1,4-glucan derivative of claim 16 or 17.

27. A heparin alternative material obtainable from a processes comprises:
introducing step of carboxyl group in which an enzymatically synthesized α-1,4-glucan is reacted with a dicarboxylic acid to introduce carboxyl group into the enzymatically synthesized α-1,4-glucan, and
introducing step of sulfonic acid group in which an enzymatically synthesized α-1,4-glucan derivative having carboxyl group prepared in the introducing step of carboxyl group is reacted with a compound having amino group and sulfonic acid group to introduce sulfonic acid group into all or a part of the carboxyl group.
